(19) United States
(12) Patent Application Publication
(10) Pub. No.: US 2024/0257929 A1
(43) Pub. Date: Aug. 1, 2024

(54) MEDICAL INFORMATION PROCESSING APPARATUS, METHOD FOR OPERATING MEDICAL INFORMATION PROCESSING APPARATUS, AND MEDICAL INFORMATION PROCESSING PROGRAM

(71) Applicant: FUJIFILM Corporation, Tokyo (JP)

(72) Inventor: Akimichi Ichinose, Tokyo (JP)

(73) Assignee: FUJIFILM Corporation, Tokyo (JP)

(21) Appl. No.: 18/416,782

(22) Filed: Jan. 18, 2024

(30) Foreign Application Priority Data
Jan. 31, 2023 (JP) 2023-013097

(51) Int. Cl. G16H 10/60; G06F 40/40

(52) U.S. Cl. CPC G16H 10/60; G06F 40/40

(57) ABSTRACT

A medical information processing apparatus includes a processor and one or more memories that store a program to be executed by the processor. The processor acquires each of an interpretation report and an electronic medical record created for the same patient, extracts one or more first lesion information items from finding information described in the interpretation report, extracts a plurality of second lesion information items from diagnosis and treatment information described in the electronic medical record, associates the one or more first lesion information items with the second lesion information indicating the same lesion as the one or more first lesion information items among the plurality of second lesion information items, and link a first description portion related to the associated first lesion information and a second description portion related to the associated second lesion information.

### CROSS-REFERENCE TO RELATED APPLICATION

The present application claims priority under 35 U.S.C § 119(a) to Japanese Patent Application No. 2023-013097 filed on Jan. 31, 2023, which is hereby expressly incorporated by reference, in its entirety, into the present application.

### BACKGROUND OF THE INVENTION

### 1. Field of the Invention

The present invention relates to a medical information processing apparatus, a method for operating a medical information processing apparatus, and a medical information processing program, and particularly relates to a technique that processes medical information in an interpretation report and an electronic medical record.

### 2. Description of the Related Art

In some cases, a radiologist creates an interpretation report with reference to a past interpretation report and diagnosis and treatment information (for example, clinical findings, a treatment history, and results of a pathological test) in an electronic medical record, in addition to medical images, and an attending physician compares the interpretation report and the diagnosis and treatment information in the electronic medical record to determine a treatment policy.

However, it takes a lot of time and effort for the radiologist to search for the diagnosis and treatment information in the electronic medical record to be referred to for creating the interpretation report, and it takes a lot of time and effort for the attending physician to search for findings to be referred to from the interpretation report. Therefore, there is a burden in a case where an image diagnosis is performed and the treatment policy is determined.

JP2020-154630A discloses a medical information collection apparatus that collects learning data used for machine learning such as deep learning.

In a case where an input indicating a history of content of diagnosis and treatment is received, the medical information collection apparatus disclosed in JP2020-154630A acquires electronic medical record information matched with the input history from a database that manages electronic medical record information indicating records of the content of the diagnosis and treatment for each patient and collects medical images specified in an interpretation report associated with the acquired electronic medical record information.

Further, JP2019-020929A discloses a hospital information system that prompts a medical worker to confirm the content of an interpretation report.

The hospital information system disclosed in JP2019-020929A registers test information and findings included in the interpretation report created in a case where a patient is tested in a database in association with a test result number. In a case where a request to display information of the interpretation report is received from the medical worker, the hospital information system searches for test information and findings corresponding to the display request from the database and acquires them. Then, the hospital information system determines whether or not the acquired findings include a predetermined keyword and displays a warning related to the keyword together with the findings and test information to be displayed in a case where the keyword is included.

### SUMMARY OF THE INVENTION

JP2020-154630A discloses the association between the interpretation report and the electronic medical record information, but does not disclose the link between a description of the findings in the interpretation report and a description of the electronic medical record information indicating the content of the diagnosis and treatment.

Further, JP2019-020929A discloses that the request to display the interpretation report linked for each patient is received from a screen of the electronic medical record, but does not disclose the link between a description of the findings in the interpretation report and a description of diagnosis and treatment information in the electronic medical record.

An embodiment according to the technology of the present disclosure provides a medical information processing apparatus, a method for operating a medical information processing apparatus, and a medical information processing program that can mutually utilize finding information described in an interpretation report and diagnosis and treatment information described in an electronic medical record.

According to a first aspect of the present invention, there is provided a medical information processing apparatus comprising: a processor; and one or more memories that store a program to be executed by the processor. The processor is configured to: acquire each of an interpretation report and an electronic medical record created for the same patient; extract one or more first lesion information items from finding information described in the interpretation report; extract a plurality of second lesion information items from diagnosis and treatment information described in the electronic medical record; associate the one or more first lesion information items with the second lesion information indicating the same lesion as the one or more first lesion information items among the plurality of second lesion information items; and link a first description portion related to the associated first lesion information in the finding information of the interpretation report and a second description portion related to the associated second lesion information in the diagnosis and treatment information of the electronic medical record.

According to the first aspect of the present invention, the one or more first lesion information items are extracted from the finding information described in the interpretation report, the plurality of second lesion information items are extracted from the diagnosis and treatment information described in the electronic medical record created for the same patient, and the first lesion information and the second lesion information indicating the same lesion are associated with each other. Then, the first description portion related to the associated first lesion information in the finding information of the interpretation report and the second description portion related to the associated second lesion information in the diagnosis and treatment information of the electronic medical record are linked to each other. The first description portion of the interpretation report and the second description portion in the diagnosis and treatment information of the electronic medical record which are linked to each other are description portions related to the same lesion. A radiologist can easily acquire the description portion (second description portion) in the diagnosis and treatment information of the electronic medical record which is referred to in a case where the interpretation report is created. An attending physician can easily acquire the description portion (first description portion) in the finding information of the interpretation report which is referred to for creating the diagnosis and treatment information and for determining a diagnosis and treatment policy.

According to a second aspect of the present invention, in the medical information processing apparatus according to the first aspect, preferably, the processor is configured to store the first description portion of the interpretation report and the second description portion of the electronic medical record, which have been linked to each other, in the memory in association with each other. Therefore, it is possible to link and manage the first description portion of the interpretation report and the second description portion of the electronic medical record using the memory.

According to a third aspect of the present invention, in the medical information processing apparatus according to the first aspect or the second aspect, preferably, the processor is configured to: display the interpretation report on a display; and in a case where an input of the first description portion is received, display the second description portion of the electronic medical record linked to the first description portion on the display. Therefore, in a case where a new interpretation report is created, it is possible to refer to the second description portion of the electronic medical record linked to the first description portion of the past interpretation report of the same patient.

According to a fourth aspect of the present invention, in the medical information processing apparatus according to any one of the first to third aspects, preferably, the processor is configured to: display the electronic medical record on a display; and in a case where an input of the second description portion is received, display the first description portion of the interpretation report linked to the second description portion on the display. Therefore, in a case where diagnosis and treatment information is added to the electronic medical record or a diagnosis and treatment policy is determined, it is possible to refer to the first description portion of the interpretation report linked to the second description portion of the electronic medical record of the same patient.

According to a fifth aspect of the present invention, in the medical information processing apparatus according to any one of the first to fourth aspects, preferably, the processor is configured to: perform natural language processing on the finding information described in the interpretation report to extract one or more first keywords indicating the one or more first lesion information items; generate one or more first structured information items structured by the one or more first keywords; determine one or more first description ranges corresponding to the one or more first lesion information items on the basis of the one or more first structured information items; perform natural language processing on the diagnosis and treatment information described in the electronic medical record to extract a plurality of third keywords indicating the plurality of second lesion information items; generate a plurality of second structured information items structured by the plurality of third keywords; determine a plurality of second description ranges corresponding to the plurality of second lesion information items in the diagnosis and treatment information on the basis of the plurality of second structured information items; associate the first structured information and the second structured information indicating the same lesion among the one or more first structured information items and the plurality of second structured information items; and link the first description range related to the associated first structured information in the finding information of the interpretation report and the second description range related to the associated second structured information in the diagnosis and treatment information of the electronic medical record.

According to the fifth aspect of the present invention, the natural language processing is performed on the finding information described in the interpretation report and the diagnosis and treatment information described in the electronic medical record to extract the one or more first keywords indicating the one or more first lesion information items and the plurality of third keywords indicating the plurality of second lesion information items, respectively. The one or more first structured information items structured by the one or more first keywords and the plurality of second structured information items structured by the plurality of third keywords are generated. The first structured information and the second structured information indicating the same lesion among the one or more first structured information items and the plurality of second structured information items which have been generated are associated with each other. The first description range related to the associated first structured information in the finding information of the interpretation report and the second description range related to the associated second structured information in the diagnosis and treatment information of the electronic medical record are linked to each other. In addition, the first description range and the second description range can include the first description portion and the second description portion, respectively.

According to a sixth aspect of the present invention, in the medical information processing apparatus according to the fifth aspect, preferably, each of the first structured information and the second structured information includes one or more attribute information items among attribute information items of a lesion indicating a type, a site, a size, and a shape of the lesion.

According to a seventh aspect of the present invention, in the medical information processing apparatus according to any one of the first to sixth aspects, preferably, the processor is configured to: perform natural language processing on the finding information described in the interpretation report to extract one or more first keywords indicating the one or more first lesion information items; specify a second keyword related to the one or more first keywords for the finding information of the interpretation report and generate one or more first structured information items structured by the first keyword and the second keyword for each of the one or more first lesion information items; perform natural language processing on the diagnosis and treatment information described in the electronic medical record to extract a plurality of third keywords indicating the plurality of second lesion information items; specify a fourth keyword related to the plurality of third keywords for the diagnosis and treatment information of the electronic medical record and generate a plurality of second structured information items structured by the third keyword and the fourth keyword for each of the plurality of second lesion information items; and link the first description portion in the finding information and the second description portion in the diagnosis and treatment information on the basis of the one or more first structured information items and the plurality of second structured information items.

According to the seventh aspect of the present invention, the first structured information and the second structured information are not limited to only the first keyword indicating the first lesion information and the third keyword indicating the second lesion information, respectively. The first structured information and the second structured information are structured by the second keyword related to the first keyword and the fourth keyword related to the third keyword, respectively, and the first description portion in the finding information and the second description portion in the diagnosis and treatment information are linked to each other on the basis of the one or more first structured information items and the plurality of second structured information items which have been structured.

According to an eighth aspect of the present invention, in the medical information processing apparatus according to the seventh aspect, preferably, each of the first structured information and the second structured information includes one or more attribute information items among attribute information items of a lesion indicating a type, a site, a size, and a shape of the lesion.

According to a ninth aspect of the present invention, in the medical information processing apparatus according to any one of the first to eighth aspects, preferably, the processor is configured to: acquire a medical image referred to in a case where the interpretation report is created; detect a lesion from the medical image and specify information related to a type and a position of the detected lesion; and link finding information related to the lesion in the interpretation report, diagnosis and treatment information related to the lesion in the electronic medical record, and the lesion in the medical image on the basis of the specified information related to the type and the position of the lesion.

According to a tenth aspect of the present invention, in the medical information processing apparatus according to the ninth aspect, preferably, the processor is configured to: display the medical image on a display; and in a case where an input indicating the lesion in the medical image is received, display the finding information and the diagnosis and treatment information linked to the lesion to be highlighted.

According to an eleventh aspect of the present invention, there is provided a method for operating a medical information processing apparatus including a processor and one or more memories that store a program to be executed by the processor. The method comprises causing the processor to: acquire each of an interpretation report and an electronic medical record created for the same patient; extract one or more first lesion information items from finding information described in the interpretation report; extract a plurality of second lesion information items from diagnosis and treatment information described in the electronic medical record; associate the one or more first lesion information items with the second lesion information indicating the same lesion as the one or more first lesion information items among the plurality of second lesion information items; and link a first description portion related to the associated first lesion information in the finding information of the interpretation report and a second description portion related to the associated second lesion information in the diagnosis and treatment information of the electronic medical record.

According to a twelfth aspect of the present invention, in the method for operating a medical information processing apparatus according to the eleventh aspect, preferably, the processor is configured to store the first description portion of the interpretation report and the second description portion of the electronic medical record, which have been linked to each other, in the memory in association with each other.

According to a thirteenth aspect of the present invention, in the method for operating a medical information processing apparatus according to the eleventh aspect or the twelfth aspect, preferably, the processor is configured to: display the interpretation report on a display; and in a case where an input of the first description portion is received, display the second description portion of the electronic medical record linked to the first description portion on the display.

According to a fourteenth aspect of the present invention, in the method for operating a medical information processing apparatus according to any one of the eleventh to thirteenth aspects, preferably, the processor is configured to: display the electronic medical record on a display; and in a case where an input of the second description portion is received, display the first description portion of the interpretation report linked to the second description portion on the display.

According to a fifteenth aspect of the present invention, there is provided a medical information processing program causing a computer to implement: a function of acquiring each of an interpretation report and an electronic medical record created for the same patient; a function of extracting one or more first lesion information items from finding information described in the interpretation report; a function of extracting a plurality of second lesion information items from diagnosis and treatment information described in the electronic medical record; a function of associating the one or more first lesion information items with the second lesion information indicating the same lesion as the one or more first lesion information items among the plurality of second lesion information items; and a function of linking a first description portion related to the associated first lesion information in the finding information of the interpretation report and a second description portion related to the associated second lesion information in the diagnosis and treatment information of the electronic medical record.

According to the aspects of the present invention, the first description portion of the finding information described in the interpretation report and the second description portion of the diagnosis and treatment information described in the electronic medical record which are related to the same lesion are linked to each other. Therefore, it is possible to easily check a desired description portion and to mutually utilize the finding information described in the interpretation report and the diagnosis and treatment information described in the electronic medical record.

### BRIEF DESCRIPTION OF THE DRAWINGS

FIG. 1 is a block diagram illustrating an embodiment of a hardware configuration of an electronic medical record system including a medical information processing apparatus according to the present invention.

FIG. 2 is a diagram illustrating an example of a screen image of an electronic medical record displayed on a display of a diagnosis and treatment department terminal.

FIG. 3 is a diagram illustrating an example of a screen image of an interpretation report displayed on a display of a radiology department terminal.

FIG. 4 is a block diagram illustrating a hardware configuration of the medical information processing apparatus according to the present invention.

FIG. 5 is a diagram illustrating an outline of content of a process by the medical information processing apparatus according to the present invention.

FIG. 6 is a diagram illustrating a first embodiment of the medical information processing apparatus and is a diagram particularly illustrating diagnosis and treatment information of an electronic medical record and finding information of an interpretation report.

FIG. 7 is a diagram in which description ranges corresponding to lesion information in the diagnosis and treatment information of the electronic medical record and the finding information of the interpretation report illustrated in FIG. 6 are represented by dotted lines.

FIG. 8 is a diagram illustrating the first embodiment of the medical information processing apparatus and is a diagram particularly illustrating attribute information related to a lesion extracted from the description range of the diagnosis and treatment information of the electronic medical record and the description range of the finding information of the interpretation report.

FIG. 9 is a diagram illustrating the first embodiment of the medical information processing apparatus and is a conceptual diagram particularly illustrating the description range of the diagnosis and treatment information of the electronic medical record and the description range of the finding information of the interpretation report which are linked to each other.

FIG. 10 is a diagram illustrating a process of a main portion of a second embodiment of the medical information processing apparatus.

FIG. 11 is a flowchart illustrating an embodiment of a method for operating a medical information processing apparatus according to the present invention.

FIG. 12 is a flowchart illustrating an embodiment in which a radiologist uses the diagnosis and treatment information of the electronic medical record to create an interpretation report.

FIG. 13 is a flowchart illustrating an embodiment in which a physician uses the finding information of the interpretation report.

FIG. 14 is a list illustrating the presence or absence of a diagnosis and treatment record, a pathological test, an interpretation report, and an administration record for each disease name of the same patient.

### DESCRIPTION OF THE PREFERRED EMBODIMENTS

Hereinafter, preferred embodiments of a medical information processing apparatus, a method for operating a medical information processing apparatus, and a medical information processing program according to the present invention will be described with reference to the accompanying drawings.

### Hardware Configuration of Electronic Medical Record System

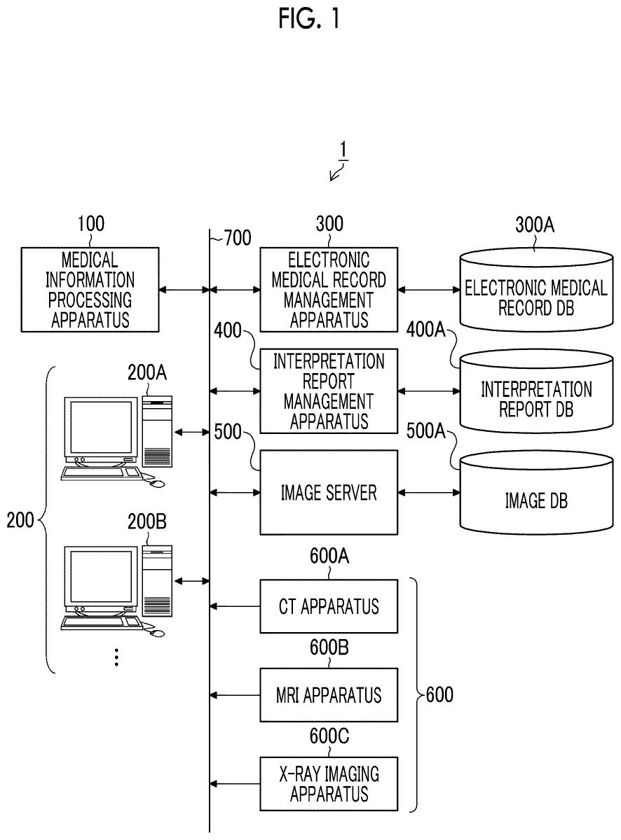

FIG. 1 is a block diagram illustrating an embodiment of a hardware configuration of an electronic medical record system including the medical information processing apparatus according to the present invention.

An electronic medical record system 1 illustrated in FIG. 1 is, for example, a system that is applied to a general hospital including a radiology department and the like and includes a medical information processing apparatus 100, a plurality of terminals 200 including a diagnosis and treatment department terminal 200A in another diagnosis and treatment department except for the radiology department and a radiology department terminal 200B, an electronic medical record management apparatus 300, an interpretation report management apparatus 400, an image server 500, and various modalities 600, such as a computed tomography (CT) apparatus 600A, a magnetic resonance imaging (MRI) apparatus 600B, and an X-ray imaging apparatus 600C, which are connected to one another through a network 700 such as a local area network (LAN).

Further, in this example, the electronic medical record management apparatus 300 comprises an electronic medical record DB 300A, the interpretation report management apparatus 400 comprises an interpretation report DB 400A, and the image server 500 comprises an image DB 500A. In addition, some or all of the electronic medical record DB 300A, the interpretation report DB 400A, and the image DB 500A may be provided in, for example, an external data center that is accessible through the network 700 to construct a cloud-type electronic medical record system.

The medical information processing apparatus 100 can be configured by, for example, a personal computer or a workstation. The medical information processing apparatus 100 according to this example is provided separately from the plurality of terminals 200. However, some or all of the plurality of terminals 200 may have the functions of the medical information processing apparatus 100.

The diagnosis and treatment department terminal 200A comprises a computer main body, a user interface consisting of a display that displays various types of information and an operation unit such as a keyboard or a mouse. For example, the following functions are incorporated into the diagnosis and treatment department terminal 200A: an electronic medical record creation function that allows a physician (attending physician) to use the user interface to record diagnosis and treatment information, such as the medical examination, test, and treatment (for example, medication) histories of a patient, as an electronic medical record; a function of reading out an interpretation report created by a radiologist, which is managed in the interpretation report DB 400A, through the interpretation report management apparatus 400 and referring to the interpretation report; and a test ordering function of creating test order information and transmitting the test order information to an ordering system (not illustrated) in the radiology department.

A processor of the diagnosis and treatment department terminal 200A executes a program that has been installed in advance to implement these functions.

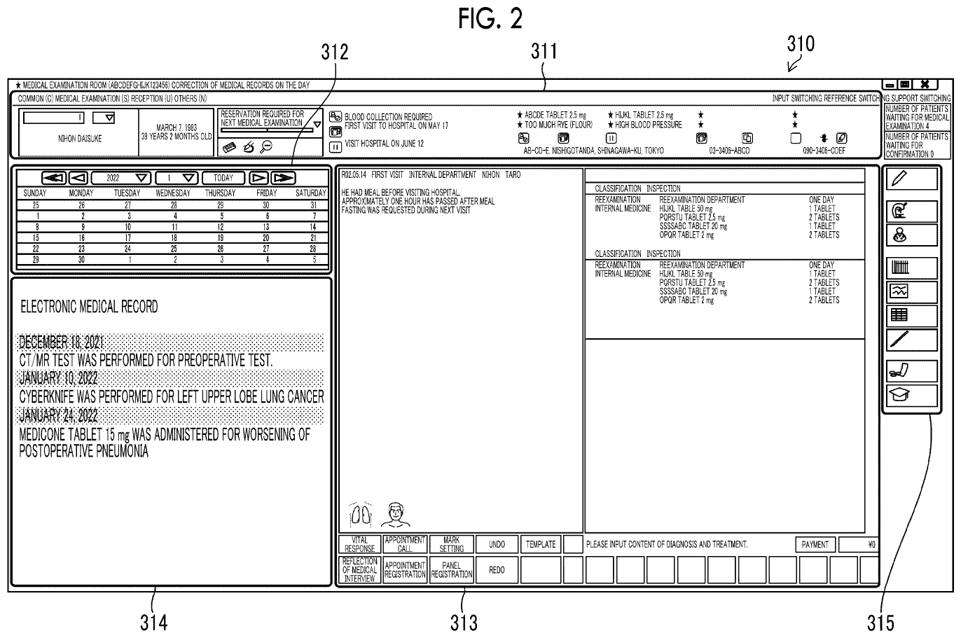

FIG. 2 is a diagram illustrating an example of a screen image of the electronic medical record displayed on the display of the diagnosis and treatment department terminal.

A screen 310 of an electronic medical record displayed on the display of the diagnosis and treatment department terminal 200A is provided with a basic information field 311 in which the name, birth date, age, gender, contact information, and the like of a patient are displayed, a diagnosis and treatment history field 312 in which a diagnosis and treatment history for each diagnosis and treatment date is displayed, a today's medical record and input field 313 to which today's diagnosis and treatment information is input, a past medical record field 314 in which past diagnosis and treatment information and the like are displayed, a menu field 315, and the like as illustrated in FIG. 2. In addition, the diagnosis and treatment history field 312 and the past medical record field 314 are configured such that the diagnosis and treatment history, the past diagnosis and treatment information, and the like can be displayed to be scrolled, respectively.

The physician uses the user interface of the diagnosis and treatment department terminal 200A to input the today's diagnosis and treatment information of the patient to the today's medical record and input field 313. In addition, as necessary, the physician can input an instruction to display the interpretation report created by the radiologist on the screen 310 in a pop-up manner, an instruction to switch and display the screen, or an instruction to display a medical image to be interpreted in the interpretation report and can use the displayed information as a reference for inputting the medical examination or diagnosis and treatment information of the patient.

In a case where the input of the today's diagnosis and treatment information in the electronic medical record is ended, in order to update the electronic medical record of the patient corresponding to the input electronic medical record, the diagnosis and treatment department terminal 200A transmits the today's diagnosis and treatment information and the like to the electronic medical record management apparatus 300 through the network 700 and requests the registration of the diagnosis and treatment information and the like in the electronic medical record DB 300A. In a case where the request to register the diagnosis and treatment information and the like is received, the electronic medical record management apparatus 300 registers the electronic medical record in which the diagnosis and treatment information and the like have been updated in the electronic medical record DB 300A.

The radiology department terminal 200B comprises a computer main body, a user interface consisting of a display that displays various types of information and an operation unit, such as a keyboard or a mouse, similarly to the diagnosis and treatment department terminal 200A and has various functions of requesting the image server 500 to browse the medical image to be interpreted in response to an input from the radiologist through the operation unit, displaying the medical image received from the image server 500 on the display, requesting the interpretation report management apparatus 400 to register the created interpretation report, and displaying the interpretation report received from the interpretation report management apparatus 400 on the display.

The radiologist interprets the medical image to be interpreted which is displayed on the display of the radiology department terminal 200B and creates an interpretation report using the user interface. In addition, in a case where the radiologist creates the interpretation report, the radiologist can use a support function of the radiology department terminal 200B which automatically detects a lesion candidate region in the medical image and which displays the lesion candidate region to be highlighted, or can input an instruction to display the diagnosis and treatment information and the like in the electronic medical record of the patient, for whom the interpretation report is ordered, on the display and refer to the displayed information.

The processor of the radiology department terminal 200B executes the program installed in advance to implement each of the above-described functions of the radiology department terminal 200B.

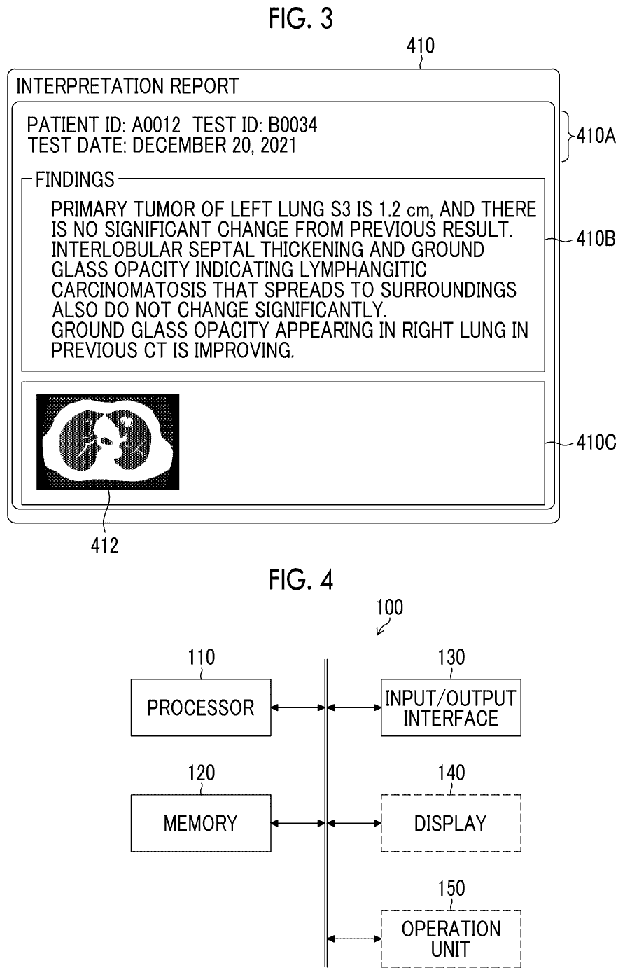

FIG. 3 is a diagram illustrating an example of a screen image of the interpretation report displayed on the display of the radiology department terminal.

A screen 410 of the interpretation report displayed on the display of the radiology department terminal 200B is provided with an accessory information field 410A in which accessory information attached to the medical image to be interpreted, such as a patient identification (ID), a test ID, and a test date, is displayed, a finding field 410B in which findings are displayed, an image field 410C in which a minified image 412 of the medical image to be interpreted is displayed, and the like as illustrated in FIG. 3. Link information to the medical image which is the original image can be attached to the minified image 412. In a case where the minified image 412 is clicked, the radiology department terminal 200B can display the medical image on the screen of the display in the pop-up manner or separately display the interpretation report and the medical image on two screens.

The radiologist uses the user interface of the radiology department terminal 200B to input character information (finding information) indicating the findings of the radiologist who has interpreted the medical image to the finding field 410B.

In the example of the interpretation report illustrated in FIG. 3, the medical image to be interpreted is a CT image of a chest, and the finding information includes attribute information of a lesion indicating the type, position, size, and the like of the lesion interpreted from the CT image of the chest. The CT image of the chest is an image obtained by imaging the chest in a cross-sectional direction with the CT apparatus 600A while changing a position in a body axis direction.

The existing terminals also have the functions of the diagnosis and treatment department terminal 200A and the radiology department terminal 200B.

### Medical Information Processing Apparatus

FIG. 4 is a block diagram illustrating a hardware configuration of the medical information processing apparatus according to the present invention.

The medical information processing apparatus 100 illustrated in FIG. 4 constitutes the electronic medical record system 1 illustrated in FIG. 1, is configured by a personal computer, a workstation, or the like, and comprises a processor 110, a memory 120, an input/output interface 130, a display 140, an operation unit 150, and the like.

The processor 110 is composed of a central processing unit (CPU) and the like, controls the overall operation of each unit of the medical information processing apparatus 100, and executes the medical information processing program stored in the memory 120 to implement various functions which will be described below.

The memory 120 is one or more memories including a flash memory, a read-only memory (ROM), a random access memory (RAM), a hard disk apparatus, and the like. The flash memory, the ROM, and the hard disk apparatus are non-volatile memories that store, for example, various programs including an operation system and the medical information processing program according to the present invention.

The RAM functions as a work area for processing by the processor 110 and temporarily stores, for example, the medical information processing program stored in the non-volatile memory, and the interpretation report and the electronic medical record created for the same patient which have been acquired (read out) from the electronic medical record DB 300A and the interpretation report DB 400A, respectively. In addition, a portion (RAM) of the memory 120 may be provided in the processor 110.

The input/output interface 130 includes, for example, a communication unit that can be connected to the network 700 and a connection unit that can be connected to an external apparatus. For example, a universal serial bus (USB) or a high-definition multimedia interface (HDMI) (HDMI is a registered trademark) can be applied as the connection unit that can be connected to the external apparatus.

The processor 110 can communicate between the electronic medical record management apparatus 300 and the interpretation report management apparatus 400 through the input/output interface 130 and the network 700 to acquire the interpretation report and the electronic medical record created for the same patient from the electronic medical record DB 300A and the interpretation report DB 400A, respectively, and can acquire the medical information processing program to be stored in the memory 120 from a server (not illustrated), which provides the medical information processing program, on the Internet.

In a case where the medical information processing apparatus 100 is also used as the diagnosis and treatment department terminal 200A or the radiology department terminal 200B, the display 140 functions as the display of the diagnosis and treatment department terminal 200A or the radiology department terminal 200B as described above. In addition, the display 140 and the operation unit 150 function as the user interface of the diagnosis and treatment department terminal 200A or the radiology department terminal 200B.

Further, the medical information processing apparatus 100 has a function of linking a description portion (first description portion) of the finding information described in the interpretation report related to the same lesion to a description portion (second description portion) of the diagnosis and treatment information described in the electronic medical record, which will be described below. The display 140 and the operation unit 150 can be used as a user interface in a case where the user (doctor) links the first description portion of the finding information to the second description portion of the diagnosis and treatment information.

In addition, in the case of the medical information processing apparatus 100 having the minimum functions, the display 140 and the operation unit 150 can be omitted.

### Outline of Medical Information Processing Apparatus

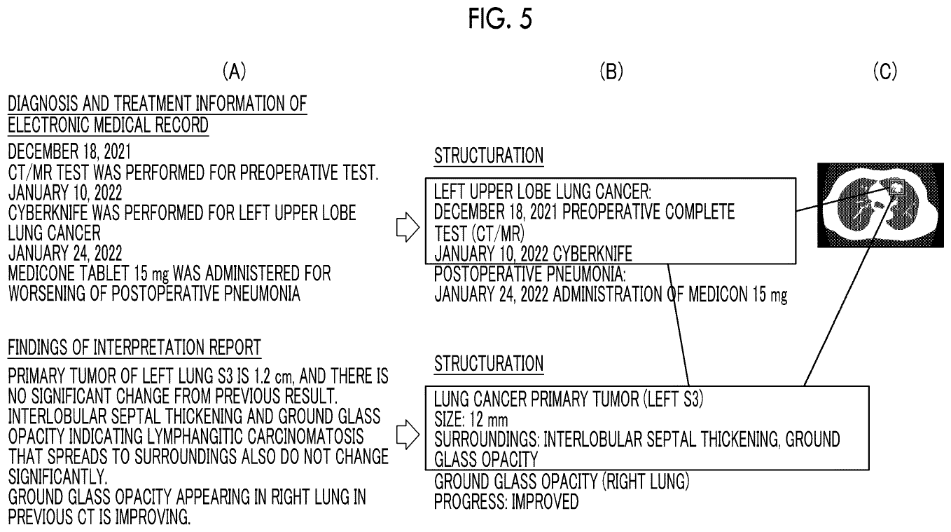

FIG. 5 is a diagram illustrating an outline of content of a process by the medical information processing apparatus according to the present invention.

The medical information processing apparatus 100 acquires the electronic medical record and the interpretation report of the same patient ((A) of FIG. 5).

The medical information processing apparatus 100 arranges (structures) the diagnosis and treatment information of the electronic medical record and the finding information of the interpretation report for each lesion, determines the relationship between the arranged information items (whether or not the descriptions are related to the same lesion), and links and manages the descriptions related to the same lesion ((B) of FIG. 5).

Furthermore, the medical image to be interpreted in the interpretation report may be separately linked ((C) of FIG. 5).

In a case where, of the descriptions linked to each other for the same lesion, the description of the interpretation report is clicked, the description of the diagnosis and treatment information of the electronic medical record is displayed. In a case where the description of the diagnosis and treatment information of the electronic medical record is clicked, the description of the interpretation report is displayed.

In general, the radiologist performs image diagnosis with reference to the past interpretation report and the diagnosis and treatment information (for example, clinical findings, a treatment history, and results of a pathological test) in the electronic medical record, and the physician also determines a treatment policy with reference to both the past interpretation report and the diagnosis and treatment information. However, the description of the diagnosis and treatment information (a diagnosis and treatment history and a pathology report) in the electronic medical record and the description of the finding information in the interpretation report are not linked to each other, which causes a burden in a case where the radiologist performs the image diagnosis and in a case where the physician determines the treatment policy.

According to the present invention, the description of the diagnosis and treatment information in the electronic medical record and the description of the finding information in the interpretation report for the same lesion are linked to each other. Therefore, it is possible to easily check a desired description portion and to mutually utilize the finding information described in the interpretation report and the diagnosis and treatment information described in the electronic medical record.

### First Embodiment of Medical Information Processing Apparatus

FIGS. 6 to 9 are diagrams illustrating a first embodiment of the medical information processing apparatus according to the present invention and particularly illustrate content of the process of the processor 110 illustrated in FIG. 4.

For example, in a case where the radiologist creates an interpretation report using the radiology department terminal 200B and registers the created interpretation report in the interpretation report DB 400A through the interpretation report management apparatus 400, the processor 110 acquires the interpretation report and acquires the electronic medical record of the same patient.

FIG. 6 is a diagram illustrating the diagnosis and treatment information of the electronic medical record and the finding information of the interpretation report. (A1) of FIG. 6 illustrates the diagnosis and treatment information of the electronic medical record, and (A2) of FIG. 6 illustrates the finding information of the interpretation report.

The diagnosis and treatment information of the electronic medical record illustrated in (A1) of FIG. 6 can be the diagnosis and treatment information in a plurality of latest diagnosis and treatment histories among the diagnosis and treatment information items of the past medical records. This is because the diagnosis and treatment information of the old past medical record is not related to the finding information of a new interpretation report.

The finding information of the interpretation report illustrated in (A2) of FIG. 6 is character information indicating the findings of the radiologist input to the finding field 410B by the radiologist (see FIG. 3).

In a case where the diagnosis and treatment information of the electronic medical record and the finding information of the interpretation report for the same patient are acquired, the processor 110 extracts one or more lesion information items (first lesion information items) from the finding information of the interpretation report and extracts a plurality of lesion information items (second lesion information items) from the diagnosis and treatment information of the electronic medical record.

Specifically, the processor 110 inputs the finding information described in the interpretation report and performs the natural language processing (unique expression extraction) on the finding information to extract one or more keywords (first keywords) related to the lesion (first lesion information). Similarly, the processor 110 inputs the diagnosis and treatment information described in the electronic medical record and performs the natural language processing on the diagnosis and treatment information to extract a plurality of keywords (third keywords) related to the lesion (second lesion information).

Therefore, it is preferable that the medical information processing program executed by the processor 110 includes a natural language processing program. In particular, it is preferable that the natural language processing program is a program suitable for extracting keywords related to lesion information.

The one or more first keywords extracted from the finding information of the interpretation report illustrated in (A2) of FIG. 6 are a “primary tumor” and “ground glass opacity” surrounded by solid frames and correspond to the one or more first lesion information items.

In addition, the plurality of third keywords extracted from the diagnosis and treatment information of the electronic medical record illustrated in (A1) of FIG. 6 are “left upper lobe lung cancer” and “postoperative pneumonia” surrounded by solid frames and correspond to the plurality of second lesion information items.

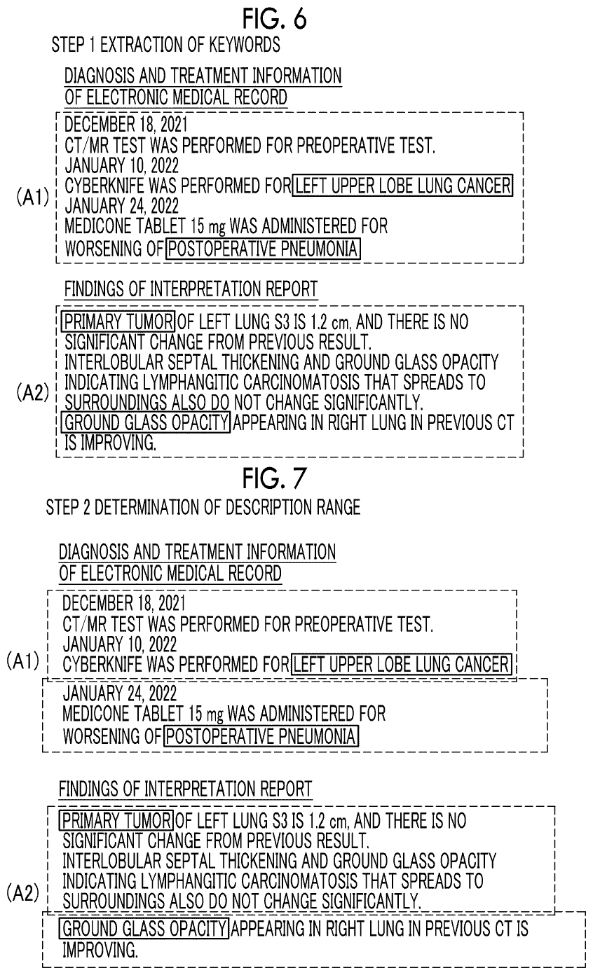

FIG. 7 is a diagram in which description ranges corresponding to the lesion information in the diagnosis and treatment information of the electronic medical record and the finding information of the interpretation report illustrated in FIG. 6 are represented by dotted lines.

The processor 110 determines one or more description ranges (one or more first description ranges) corresponding to the one or more first lesion information items in the finding information of the interpretation report on the basis of the one or more extracted first keywords. Similarly, the processor 110 determines a plurality of description ranges (a plurality of second description ranges) corresponding to the plurality of second lesion information items in the diagnosis and treatment information of the electronic medical record on the basis of the plurality of extracted third keywords.

In the example illustrated in (A1) of FIG. 7, in the diagnosis and treatment information of the electronic medical record, a description range corresponding to the “left upper lobe lung cancer” and a description range corresponding to the “postoperative pneumonia” are determined, and two description ranges are represented by dotted frames.

Further, in the example illustrated in (A2) of FIG. 7, in the finding information of the interpretation report, a description range corresponding to the “primary tumor” and a description range corresponding to the “ground glass opacity” are determined, and two description ranges are represented by dotted frames.

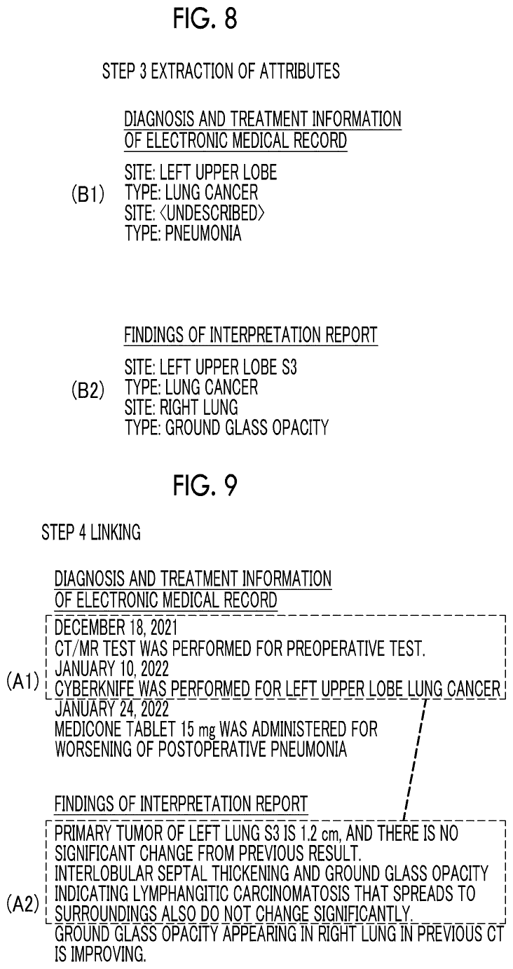

FIG. 8 is a diagram illustrating attribute information related to the lesions extracted from the description ranges of the diagnosis and treatment information of the electronic medical record and the description ranges of the finding information of the interpretation report.

The processor 110 extracts attribute information related to the lesion for each of the determined description ranges of the diagnosis and treatment information of the electronic medical record. Similarly, the processor 110 extracts attribute information related to the lesion for each of the determined description ranges of the finding information of the interpretation report.

Here, the attribute information related to the lesion is information indicating, for example, the type, site, size, and shape of the lesion.

In the example illustrated in (B1) of FIG. 8, in the attribute information extracted from the upper description range of the two description ranges of the diagnosis and treatment information of the electronic medical record, the “site” is a “left upper lobe”, and the “type” is “lung cancer”. In the attribute information extracted from the lower description range, the “site” is “undescribed”, and the “type” is “pneumonia”.

Further, in the example illustrated in (B2) of FIG. 8, in the attribute information extracted from the upper description range of the two description ranges of the finding information of the interpretation report, the “site” is a “left upper lobe S3”, and the “type” is “lung cancer”. In the attribute information extracted from the lower description range, the “site” is a “right lung”, and the “type” is “ground glass opacity”.

The processor 110 determines which description ranges among the plurality of description ranges of the diagnosis and treatment information of the electronic medical record and among the one or more description ranges of the finding information of the interpretation report correspond to each other on the basis of the attribute information (in the example illustrated in FIG. 8, the type and site of the lesion) related to the lesion extracted from each of the plurality of description ranges of the diagnosis and treatment information of the electronic medical record and the one or more description ranges of the finding information of the interpretation report.

In this example, the attribute information extracted from the upper description range of the two description ranges in the diagnosis and treatment information of the electronic medical record is matched with the attribute information extracted from the upper description range of the two description ranges in the finding information of the interpretation report. Therefore, the processor 110 determines that the upper description range of the two description ranges in the diagnosis and treatment information of the electronic medical record corresponds to the upper description range of the two description ranges in the finding information of the interpretation report.

On the other hand, in the attribute information extracted from the lower description range of the two description ranges in the diagnosis and treatment information of the electronic medical record and the attribute information extracted from the lower description range of the two description ranges in the finding information of the interpretation report, the “types” of the lesions are not necessarily matched with each other. In addition, since it is not possible to extract the “site” of the lesion from the lower description range in the diagnosis and treatment information of the electronic medical record, the “sites” of the lesions are not matched with each other. Therefore, the processor 110 determines that the lower description range in the diagnosis and treatment information of the electronic medical record does not correspond to the lower description range in the finding information of the interpretation report.

In a case where the processor 110 determines (decides) the corresponding description range between a plurality of description ranges in the diagnosis and treatment information of the electronic medical record and one or more description ranges in the finding information of the interpretation report on the basis of the attribute information related to the lesion extracted from each description range as described above, the processor 110 links the decided description range (first description range) of the interpretation report to a description range (second description range) corresponding to the decided description range in the diagnosis and treatment information of the electronic medical record.

FIG. 9 is a conceptual diagram illustrating the description range in the diagnosis and treatment information of the electronic medical record and the description range in the finding information of the interpretation report which are linked to each other.

In the example illustrated in FIG. 9, the upper description range surrounded by the dotted line of the two description ranges in the diagnosis and treatment information of the electronic medical record illustrated in (A1) of FIG. 7 is linked to the upper description range surrounded by the dotted line of the two description ranges in the finding information of the interpretation report illustrated in (A2) of FIG. 7.

The link of the description range of the interpretation report corresponding to the description range of the electronic medical record can be performed by embedding link information indicating identification information, location information, or the like of the description range of the interpretation report (attaching a hyperlink) in information resources such as text information (for example, a keyword related to the lesion) in the description range of the electronic medical record. Similarly, the link of the description range of the electronic medical record corresponding to the description range of the interpretation report can be performed by attaching a hyperlink indicating identification information, location information, or the like of the description range of the electronic medical record to text information (for example, a keyword related to the lesion) in the description range of the interpretation report. In addition, it is preferable that the keyword to which the hyperlink has been attached is displayed in a different color or font from other characters such that the attachment of the hyperlink can be visibly recognized.

The processor 110 attaches hyperlinks to the interpretation report and the electronic medical record as described above to update each of the interpretation report and the electronic medical record and registers the updated interpretation report and the updated electronic medical record in the interpretation report DB 400A and the electronic medical record DB 300A through the interpretation report management apparatus 400 and the electronic medical record management apparatus 300, respectively. Further, the present invention is not limited to the case where the hyperlinks are attached to the interpretation report and the electronic medical record. For example, the processor 110 may store the first description range which is the first description portion of the interpretation report and the second description range which is the second description portion of the electronic medical record, which have been linked to each other, in the memory 120 in association with each other and may read out information of the description ranges related to each other as necessary.

### Second Embodiment of Medical Information Processing Apparatus

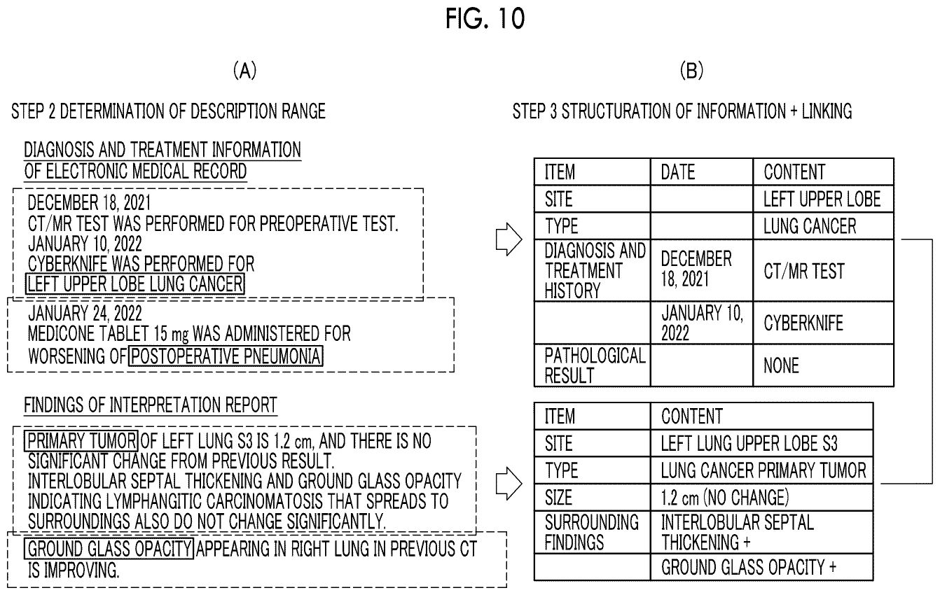

FIG. 10 is a diagram illustrating a process of a main portion of a second embodiment of the medical information processing apparatus.

The medical information processing apparatus according to the second embodiment illustrated in FIG. 10 is different from the medical information processing apparatus according to the first embodiment illustrated in FIGS. 6 to 9 in that it generates structured information which will be described below and links the description range in the finding information of the interpretation report to the description range in the diagnosis and treatment information of the electronic medical record that corresponds to the description range in the finding information on the basis of the generated structured information.

The processor 110 acquires each of the finding information of the interpretation report and the diagnosis and treatment information of the electronic medical record for the same patient and performs the natural language processing on the finding information to extract one or more first keywords related to the first lesion information. Similarly, the processor 110 performs the natural language processing on the diagnosis and treatment information to extract a plurality of third keywords related to the second lesion information. The process up to this step is the same as that in the first embodiment.

Then, the processor 110 generates one or more first structured information items structured by the one or more first keywords extracted from the finding information of the interpretation report. Similarly, the processor 110 generates a plurality of second structured information items structured by the plurality of third keywords extracted from the diagnosis and treatment information of the electronic medical record.

Here, each of the first structured information and the second structured information includes one or more attribute information items among the attribute information items of the lesion indicating the type, site, size, and shape of the lesion.

The processor 110 associates the first structured information and the second structured information indicating the same lesion among the one or more generated first structured information items and the plurality of generated second structured information items. Then, a description range (first description range) related to the associated first structured information in the finding information of the interpretation report is linked to a description range (second description range) related to the associated second structured information in the diagnosis and treatment information of the electronic medical record.

Next, a specific example of a method for generating the first structured information and the second structured information will be described.

The processor 110 performs the natural language processing on the finding information described in the interpretation report to extract one or more first keywords indicating one or more first lesion information items and specifies keywords (second keywords) that are related to the one or more extracted first keywords.

The second keywords related to the first keywords are not keywords indicating the first lesion information, but are keywords specified from peripheral finding information related to the first lesion information.

In the example illustrated in (B) of FIG. 10, the first keywords extracted from the finding information described in the interpretation report are a “left lung upper lobe S3” indicating the site of a lesion, a “lung cancer primary tumor” indicating the type of the lesion, and “1.2 cm (no change)” indicating the size of the lesion, and the second keywords related to the first keywords are “interlobular septal thickening” and “ground glass opacity”.

Similarly, the processor 110 performs the natural language processing on the diagnosis and treatment information described in the electronic medical record to extract a plurality of third keywords indicating a plurality of second lesion information items and specifies keywords (fourth keywords) that are related to the plurality of extracted third keywords.

In the example illustrated in (B) of FIG. 10, the third keywords extracted from the diagnosis and treatment information described in the electronic medical record is a “left lung upper lobe” indicating the site of a lesion and “lung cancer” indicating the type of the lesion, and the fourth keywords related to the third keywords are a “CT/MR test” which is a diagnosis and treatment history and “cyber knife” which is a treatment history. In addition, a pathological result is determined to be “none”.

Therefore, the present invention is not limited to the case where one or more first structured information items are generated from one or more first keywords extracted from the finding information of the interpretation report. The processor 110 can generate one or more first structured information items structured by one or more first keywords and second keywords. Similarly, the present invention is not limited to the case where a plurality of second structured information items are generated from a plurality of third keywords extracted from the diagnosis and treatment information of the electronic medical record. The processor 110 can generate a plurality of second structured information items structured by a plurality of third keywords and fourth keywords.

The processor 110 can associate the first structured information and the second structured information indicating the same lesion among the one or more first structured information items and the plurality of second structured information items generated in this way (see (B) of 10) and can link the first description range related to the associated first structured information in the finding information of the interpretation report to the second description range related to the associated second structured information in the diagnosis and treatment information of the electronic medical record.

### Third Embodiment of Medical Information Processing Apparatus

A third embodiment of the medical information processing apparatus according to the present invention is different from the first embodiment and the second embodiment in that information related to the type and position of a lesion is specified from the medical image mainly referred to in a case where the interpretation report is created and the finding information related to the lesion in the interpretation report, the diagnosis and treatment information related to the lesion in the electronic medical record, and the lesion in the medical image are linked on the basis of the specified information (attribute information) related to the type and position of the lesion.

That is, the processor 110 acquires the medical image referred to in a case where the interpretation report is created together with the interpretation report. The medical image referred to in a case where the interpretation report is created can be acquired from the interpretation report on the basis of the link information attached to the minified image 412 as illustrated in FIG. 3.

Then, the processor 110 specifies the information related to the type and position of the lesion from the acquired medical image.

The processor 110 has the function of a recognizer that recognizes the position of a lesion region included in the medical image and the type of lesion in the lesion region on the basis of the medical image. The processor 110 executes, for example, a convolution neural network (CNN) which is one of learning models, classifies (segments) a lesion region or the like in an input medical image for each pixel or each group consisting of several pixels to recognize a region (position) of a lesion, and classifies the class of the lesion in the recognized lesion region to recognize the type of the lesion.

Preferably, the processor 110 links the finding information related to the lesion in the interpretation report that includes the information related to the type and position of the same lesion to the diagnosis and treatment information related to the lesion in the electronic medical record as illustrated in (B) and (C) of FIG. 5 and further links the lesion in the medical image, on the basis of the acquired interpretation report and electronic medical record and the information related to the type and position of the lesion specified from the medical images as described above.

### Method for Operating Medical Information Processing Apparatus

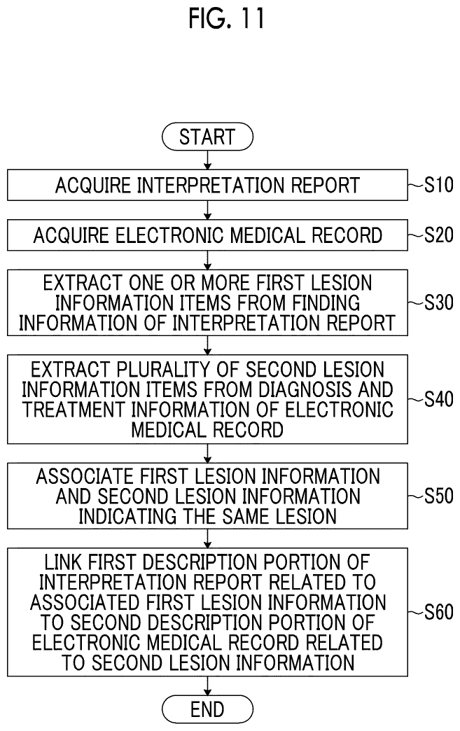

FIG. 11 is a flowchart illustrating an embodiment of a method for operating the medical information processing apparatus according to the present invention.

A process in each step of the method for operating the medical information processing apparatus illustrated in FIG. 11 corresponds to the process performed by the processor 110 of the medical information processing apparatus 100 illustrated in FIG. 4.

In FIG. 11, the processor 110 acquires each of the interpretation report and the electronic medical record created for the same patient (Step S10 and Step S20). The processor 110 may be triggered to acquire each of the interpretation report and the electronic medical record created for the same patient in a case where the radiologist creates the interpretation report and registers the created interpretation report in the interpretation report DB 400A or in a case where the physician adds new diagnosis and treatment information to the electronic medical record to update the electronic medical record and registers the updated electronic medical record in the electronic medical record DB 300A. Alternatively, the processor 110 may acquire each of the interpretation report and the electronic medical record created for the same patient in a case where the user uses the user interface to specify the interpretation report or the electronic medical record to be linked.

Then, the processor 110 extracts one or more first lesion information items from the finding information of the interpretation report (Step S30). Similarly, the processor 110 extracts a plurality of second lesion information items from the diagnosis and treatment information of the electronic medical record (Step S40). Specifically, the processor 110 inputs the finding information described in the interpretation report, performs the natural language processing on the finding information to specify one or more first keywords related to the lesion, and extracts one or more first lesion information items (see (A2) of FIG. 6). Similarly, the processor 110 inputs the diagnosis and treatment information described in the electronic medical record, performs the natural language processing on the diagnosis and treatment information to specify a plurality of third keywords related to the lesion, and extracts a plurality of second lesion information items (see (A1) of FIG. 6).

Then, the processor 110 associates the first lesion information and the second lesion information indicating the same lesion among the one or more extracted first lesion information items and the plurality of extracted second lesion information items (Step S50). In the example illustrated in FIG. 6, the “primary tumor of the left lung S3” extracted from the finding information of the interpretation report and the “left upper lobe lung cancer” extracted from the diagnosis and treatment information of the electronic medical record are lesion information items (the first lesion information and the second lesion information) having the same position and type of the lesion. Therefore, the first lesion information and the second lesion information are associated with each other.

The processor 110 links the first description portion related to the associated first lesion information in the finding information of the interpretation report to the second description portion related to the associated second lesion information in the diagnosis and treatment information of the electronic medical record on the basis of the first lesion information and the second lesion information associated with each other (Step S60). The first description portion and the second description portion can be linked to each other by attaching, to each description portion, link information on the other description portion.

### Embodiment in Which Radiologist Uses diagnosis and Treatment Information of Electronic Medical Record

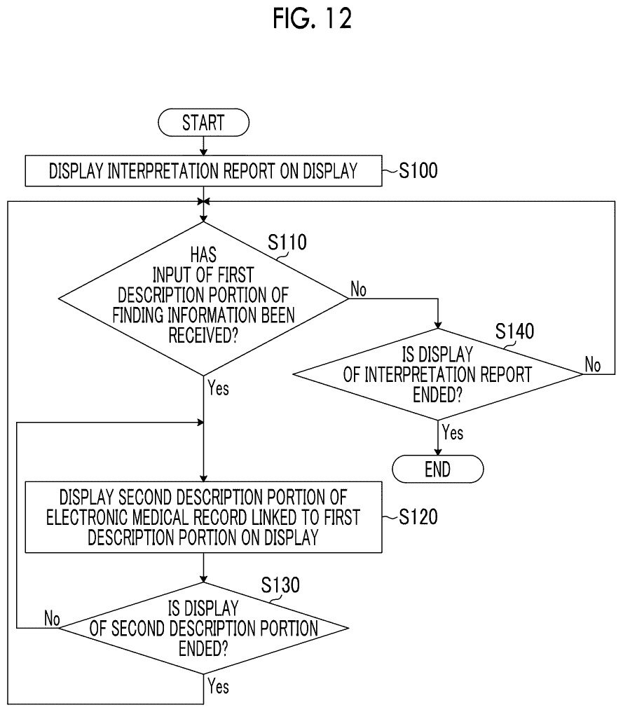

FIG. 12 is a flowchart illustrating an embodiment in which the radiologist uses the diagnosis and treatment information of the electronic medical record in a case where the radiologist creates an interpretation report.

In some cases, the radiologist creates an interpretation report with reference to the past interpretation report of the same patient and the diagnosis and treatment information (for example, clinical findings, a treatment history, and results of a pathological test) in the electronic medical record, in addition to the medical image captured by the modality 600. A case will be described in which the radiologist refers to the diagnosis and treatment information in the electronic medical record.

In a case where the radiologist creates a new interpretation report using the radiology department terminal 200B, the radiologist inputs an instruction to display the past interpretation report of the same patient on the display of the radiology department terminal 200B in addition to the new interpretation report (Step S100).

In a case where the radiologist refers to the description portion (second description portion) of the diagnosis and treatment information described in the electronic medical record which is linked to the description portion (first description portion) related to the lesion in the finding information described in the past interpretation report displayed on the display, the radiologist clicks the first description portion of the finding information.

The radiology department terminal 200B determines whether or not the input (clicking) of the first description portion of the finding information has been received (Step S110) and displays the second description portion of the electronic medical record linked to the first description portion on the display in a case where the input has been received (Step S120). It is preferable that the second description portion is displayed on the display to be highlighted by, for example, pop-up display or highlight display.

For example, in the example illustrated in FIG. 9, in a case where the first description portion ((A2) of FIG. 9) surrounded by a dotted line in the finding information of the interpretation report is clicked, the second description portion ((A1) of FIG. 9) surrounded by a dotted line in the diagnosis and treatment information of the electronic medical record which is linked to the first description portion is displayed on the display in the pop-up manner.

In addition, since a hyperlink to the second description portion of the electronic medical record is attached to the first description portion (or the first lesion information included in the first description portion) of the interpretation report, it is possible to read out the second description portion of the electronic medical record according to the hyperlink and to display the second description portion on the display in the pop-up manner.

Then, the radiology department terminal 200B determines whether or not to end the display (pop-up display) of the second description portion of the electronic medical record, and the process proceeds to Step S110 in a case where it is determined to end the display (Step S130). In addition, the display of the second description portion can be ended in a case where a certain time has elapsed since the pop-up display or in a case where an input, such as clicking, is made again.

On the other hand, in a case where the input of the first description portion of the finding information has not been received in Step S110, the process proceeds to Step S140 to determine whether or not to end the display of the interpretation report referred to here (Step S140). The determination of whether or not to end the display of the interpretation report referred to can be performed on the basis of the operation of the radiology department terminal 200B.

In a case where it is determined to end the display of the interpretation report, the radiology department terminal 200B ends the display of the interpretation report referred to, and the process proceeds to Step S110 in a case where it is determined not to end the display of the interpretation report.

Therefore, the radiologist can single-click the desired first description portion related to the lesion in the finding information described in the past interpretation report displayed on the display of the radiology department terminal 200B to display the second description portion described in association with the same lesion in the diagnosis and treatment information described in the electronic medical record on the display in the pop-up manner, and can refer to the second description portion in the case of creating a new interpretation report.

### Embodiment in Which Physician Uses Finding Information of Interpretation Report

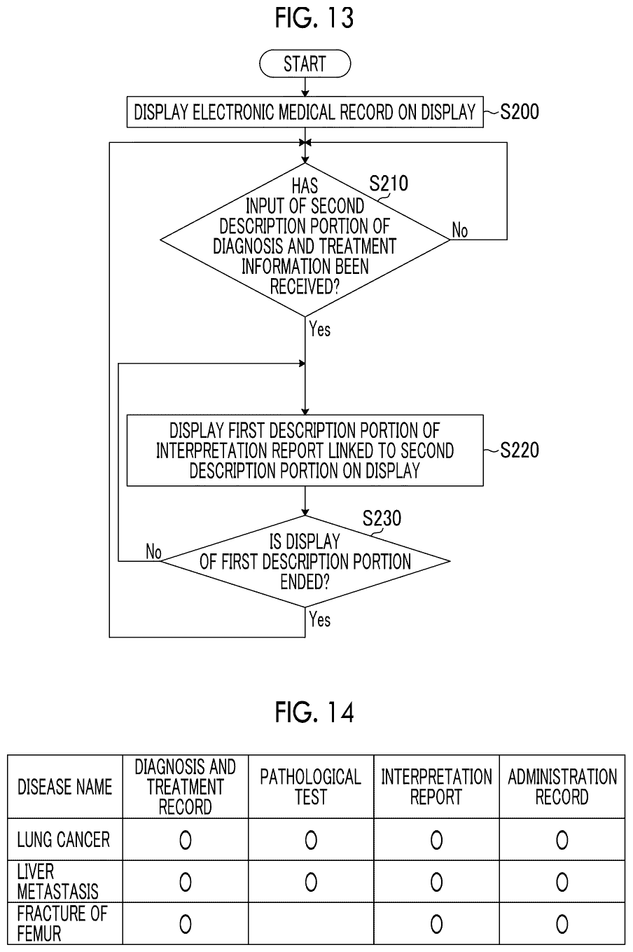

FIG. 13 is a flowchart illustrating an embodiment in which the physician uses the finding information of the interpretation report.

There are the following cases: the physician determines a treatment policy; the physician inputs diagnosis and treatment information into the electronic medical record; and the physician refers to the interpretation report in addition to the past diagnosis and treatment information (for example, clinical findings, a treatment history, and results of a pathological test) of the electronic medical record. A case will be described where the physician refers to the finding information of the interpretation report.

In a case where the physician performs, for example, the determination of the treatment policy for the patient in charge, the physician inputs an instruction to display the electronic medical record of the patient in charge on the display of the diagnosis and treatment department terminal 200A (Step S200).

In a case where the physician refers to the description portion (first description portion) of the finding information described in the interpretation report which is linked to the description portion (second description portion) related to the lesion in the diagnosis and treatment information of the electronic medical record (see FIG. 2) in the past medical record field 314 displayed on the display, the physician clicks the second description portion of the diagnosis and treatment information.

The diagnosis and treatment department terminal 200A determines whether or not the input (clicking) of the second description portion of the diagnosis and treatment information has been received (Step S210) and displays the first description portion of the interpretation report linked to the second description portion on the display in a case where the input has been received (Step S220). It is preferable that the first description portion is displayed on the display to be highlighted by, for example, pop-up display or highlight display.

For example, in the example illustrated in FIG. 9, in a case where the second description portion ((A1) of FIG. 9) surrounded by the dotted line in the diagnosis and treatment information of the electronic medical record is clicked, the first description portion ((A2) of FIG. 9) surrounded by the dotted line in the finding information of the interpretation report which is linked to the second description portion is displayed on the display in the pop-up manner.

In addition, since the hyperlink to the first description portion of the interpretation report is attached to the second description portion (or the second lesion information included in the second description portion) of the electronic medical record, it is possible to read out the first description portion of the interpretation report according to the hyperlink and to display the first description portion on the display in the pop-up manner.

Then, the diagnosis and treatment department terminal 200A determines whether or not to end the display (pop-up display) of the first description portion of the interpretation report, and the process proceeds to Step S210 in a case where it is determined to end the display (Step S230). In addition, the display of the first description portion can be ended in a case where a certain time has elapsed since the pop-up display or in a case where an input, such as clicking, is made again.

Therefore, the physician can single-click the desired second description portion related to the lesion in the diagnosis and treatment information described in the past medical record displayed on the display of the diagnosis and treatment department terminal 200A to display the first description portion described in association with the same lesion in the finding information described in the interpretation report on the display in the pop-up manner. Therefore, the physician can use the first description portion described in association with the same lesion in the finding information described in the interpretation report to determine a treatment policy or can refer to the first description portion in the case of inputting today's diagnosis and treatment information in the today's medical record and input field 313.

In addition, the diagnosis and treatment department terminal 200A can display the first description portion described in association with the same lesion in the finding information described in the interpretation report and the medical image to be interpreted in the interpretation report and can display the position of the lesion linked to the lesion on the medical image using an indicator such as a bounding box (see (C) of FIG. 5).

In this embodiment, the first description portion of the interpretation report and the second description portion of the diagnosis and treatment information of the electronic medical record which are linked to each other are not limited to the descriptions in the first description range and the second description range determined by the description range determination process. The structured information items may be linked to each other as illustrated in (B) of FIG. 10. That is, the first structured information structured by the first keywords in the first description range or the first structured information structured by the first keywords and the second keywords related to the first keywords is linked to the second structured information structured by the third keywords in the second description range or the second structured information structured by the third keywords and the fourth keywords related to the third keywords.

Then, in a case where the first description portion of the interpretation report is displayed in the pop-up manner, the first structured information (for example, the lower structured information in (B) of FIG. 10) may be displayed in the pop-up manner. Similarly, in a case where the second description portion of the electronic medical record is displayed in the pop-up manner, the second structured information (for example, the upper structured information in (B) of FIG. 10) may be displayed in the pop-up manner. Further, in this case, it is preferable that the first structured information and the second structured information which are linked to each other are stored in, for example, the memory 120 in association with each other such that they can be read out and displayed as appropriate.

### Others

In a case where a lesion is detected from a medical image, information related to the type and position of the detected lesion is specified, and finding information related to the lesion in the interpretation report, diagnosis and treatment information related to the lesion in the electronic medical record, and the lesion in the medical image are linked on the basis of the specified information related to the type and position of the lesion, the processor may display the medical image on the display. In a case where an input indicating the lesion in the medical image is received, the processor may display the finding information and the diagnosis and treatment information linked to the lesion to be highlighted.

FIG. 14 is a list table illustrating the presence or absence of a diagnosis and treatment record, a pathological test, an interpretation report, and an administration record for each disease name of the same patient.

As in the diagnosis and treatment history in the diagnosis and treatment history field 312 of the electronic medical record illustrated in FIG. 2, the medical information processing apparatus 100 may summarize the diagnosis and treatment information of the electronic medical record and the finding information of the interpretation report for each lesion and link them to each other as illustrated in FIG. 14 to implement a function of “jump to a lesion description portion in a case where a cell in the list is clicked”.

In addition, the terminal 200 including the diagnosis and treatment department terminal 200A and the radiology department terminal 200B may have the functions of the medical information processing apparatus 100 added thereto.

Further, in this embodiment, for example, a hardware structure of the processing unit performing various processes, such as the CPU, is the following various processors. The various processors include, for example, a central processing unit (CPU) which is a general-purpose processor executing software (program) to function as various processing units, a programmable logic device (PLD), such as a field programmable gate array (FPGA), which is a processor whose circuit configuration can be changed after manufacture, and a dedicated electric circuit, such as an application specific integrated circuit (ASIC), which is a processor having a dedicated circuit configuration designed to perform a specific process.

One processing unit may be configured by one of these various processors or by two or more processors of the same type or different types (for example, a combination of a plurality of FPGAs or a combination of a CPU and an FPGA). Moreover, a plurality of processing units may be configured by one processor. A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As described above, various processing units are configured by using one or more of the various processors as the hardware structure.

In addition, more specifically, the hardware structure of these various processors is an electric circuit (circuitry) obtained by combining circuit elements such as semiconductor elements.

Further, the present invention includes a medical information processing program that is installed in a computer and that causes the computer to function as the medical information processing apparatus according to the present invention and a non-volatile storage medium that has the medical information processing program recorded thereon.

Furthermore, it is needless to say that the present invention is not limited to the above-described embodiment and various modifications can be made without departing from the gist of the present invention.

### EXPLANATION OF REFERENCES

1: electronic medical record system
   100: medical information processing apparatus
   110: processor
   120: memory
   130: input/output interface
   140: display
   150: operation unit
   200: terminal
   200A: diagnosis and treatment department terminal
   200B: radiology department terminal
   300: electronic medical record management apparatus
   300A: electronic medical record DB
   310: screen
   311: basic information field
   312: diagnosis and treatment history field
   313: today's medical record and input field
   314: past medical record field
   315: menu field
   400: interpretation report management apparatus
   400A: interpretation report DB
   410: screen
   410A: accessory information field
   410B: finding field
   410C: image field
   412: minified image
   500: image server
   500A: image DB
   600: modality
   600A: CT apparatus
   600B: MRI apparatus
   600C: X-ray imaging apparatus
   700: network
   S10 to S60, S100 to S140, S200 to S230: step

What is claimed is:

1. A medical information processing apparatus comprising:
a processor; and
one or more memories that store a program to be executed by the processor,
wherein the processor is configured to:
acquire each of an interpretation report and an electronic medical record created for the same patient;
extract one or more first lesion information items from finding information described in the interpretation report;
extract a plurality of second lesion information items from diagnosis and treatment information described in the electronic medical record;
associate the one or more first lesion information items with the second lesion information indicating the same lesion as the one or more first lesion information items among the plurality of second lesion information items; and
link a first description portion related to the associated first lesion information in the finding information of the interpretation report and a second description portion related to the associated second lesion information in the diagnosis and treatment information of the electronic medical record.

2. The medical information processing apparatus according to claim 1,
wherein the processor is configured to:
store the first description portion of the interpretation report and the second description portion of the electronic medical record, which have been linked to each other, in the memory in association with each other.

3. The medical information processing apparatus according to claim 1,
wherein the processor is configured to:
display the interpretation report on a display; and
in a case where an input of the first description portion is received, display the second description portion of the electronic medical record linked to the first description portion on the display.

4. The medical information processing apparatus according to claim 1,
wherein the processor is configured to:
display the electronic medical record on a display; and
in a case where an input of the second description portion is received, display the first description portion of the interpretation report linked to the second description portion on the display.

5. The medical information processing apparatus according to claim 1,
wherein the processor is configured to:
perform natural language processing on the finding information described in the interpretation report to extract one or more first keywords indicating the one or more first lesion information items;
generate one or more first structured information items structured by the one or more first keywords;
determine one or more first description ranges corresponding to the one or more first lesion information items on the basis of the one or more first structured information items;
perform natural language processing on the diagnosis and treatment information described in the electronic medical record to extract a plurality of third keywords indicating the plurality of second lesion information items;
generate a plurality of second structured information items structured by the plurality of third keywords;
determine a plurality of second description ranges corresponding to the plurality of second lesion information items in the diagnosis and treatment information on the basis of the plurality of second structured information items;
associate the first structured information and the second structured information indicating the same lesion among the one or more first structured information items and the plurality of second structured information items; and
link the first description range related to the associated first structured information in the finding information of the interpretation report and the second description range related to the associated second structured information in the diagnosis and treatment information of the electronic medical record.

6. The medical information processing apparatus according to claim 5,
wherein each of the first structured information and the second structured information includes one or more attribute information items among attribute information items of a lesion indicating a type, a site, a size, and a shape of the lesion.

7. The medical information processing apparatus according to claim 1,
wherein the processor is configured to:
perform natural language processing on the finding information described in the interpretation report to extract one or more first keywords indicating the one or more first lesion information items;
specify a second keyword related to the one or more first keywords for the finding information of the interpretation report and generate one or more first structured information items structured by the first keyword and the second keyword for each of the one or more first lesion information items;
perform natural language processing on the diagnosis and treatment information described in the electronic medical record to extract a plurality of third keywords indicating the plurality of second lesion information items;
specify a fourth keyword related to the plurality of third keywords for the diagnosis and treatment information of the electronic medical record and generate a plurality of second structured information items structured by the third keyword and the fourth keyword for each of the plurality of second lesion information items; and
link the first description portion in the finding information and the second description portion in the diagnosis and treatment information on the basis of the one or more first structured information items and the plurality of second structured information items.

8. The medical information processing apparatus according to claim 7,
wherein each of the first structured information and the second structured information includes one or more attribute information items among attribute information items of a lesion indicating a type, a site, a size, and a shape of the lesion.

9. The medical information processing apparatus according to claim 1,
wherein the processor is configured to:
acquire a medical image referred to in a case where the interpretation report is created;
detect a lesion from the medical image and specify information related to a type and a position of the detected lesion; and
link finding information related to the lesion in the interpretation report, diagnosis and treatment information related to the lesion in the electronic medical record, and the lesion in the medical image on the basis of the specified information related to the type and the position of the lesion.

10. The medical information processing apparatus according to claim 9,
wherein the processor is configured to:
display the medical image on a display; and
in a case where an input indicating the lesion in the medical image is received, display the finding information and the diagnosis and treatment information linked to the lesion to be highlighted.

11. A method for operating a medical information processing apparatus including a processor and one or more memories that store a program to be executed by the processor, the method comprising:
causing the processor to:
acquire each of an interpretation report and an electronic medical record created for the same patient;
extract one or more first lesion information items from finding information described in the interpretation report;
extract a plurality of second lesion information items from diagnosis and treatment information described in the electronic medical record;
associate the one or more first lesion information items with the second lesion information indicating the same lesion as the one or more first lesion information items among the plurality of second lesion information items; and
link a first description portion related to the associated first lesion information in the finding information of the interpretation report and a second description portion related to the associated second lesion information in the diagnosis and treatment information of the electronic medical record.

12. The method for operating a medical information processing apparatus according to claim 11,
wherein the processor is configured to:
store the first description portion of the interpretation report and the second description portion of the electronic medical record, which have been linked to each other, in the memory in association with each other.

13. The method for operating a medical information processing apparatus according to claim 11,
wherein the processor is configured to:
display the interpretation report on a display; and
in a case where an input of the first description portion is received, display the second description portion of the electronic medical record linked to the first description portion on the display.

14. The method for operating a medical information processing apparatus according to claim 11,
wherein the processor is configured to:
display the electronic medical record on a display; and
in a case where an input of the second description portion is received, display the first description portion of the interpretation report linked to the second description portion on the display.

15. A non-transitory, computer-readable tangible recording medium which records thereon a program for causing, when read by a computer, the computer to implement:
a function of acquiring each of an interpretation report and an electronic medical record created for the same patient;
a function of extracting one or more first lesion information items from finding information described in the interpretation report;
a function of extracting a plurality of second lesion information items from diagnosis and treatment information described in the electronic medical record;
a function of associating the one or more first lesion information items with the second lesion information indicating the same lesion as the one or more first lesion information items among the plurality of second lesion information items; and
a function of linking a first description portion related to the associated first lesion information in the finding information of the interpretation report and a second description portion related to the associated second lesion information in the diagnosis and treatment information of the electronic medical record.
